(19) United States
(12) Patent Application Publication
(10) Pub. No.: US 2010/0325755 A1
(43) Pub. Date: Dec. 23, 2010

(54) PESTICIDAL COMBINATIONS

(75) Inventor: Alex Cochran, Greensboro, NC (US)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/664,073

(22) PCT Filed: Jun. 11, 2008
(86) PCT No.: PCT/EP2008/004654

§ 371 Date: Jul. 27, 2010

(30) Foreign Application Priority Data
Jun. 14, 2007 (US) 60943868

(51) Int. Cl. A01H 5/00; A01N 43/828; A01P 21/00; A01P 7/04; A01P 3/00; A01N 37/18

(52) U.S. Cl. 800/298; 504/261; 504/320

(57) ABSTRACT

A method of controlling or preventing pest and/or pathogenic damage in a plant propagation material, a plant, and/or plant organs that grow at a later point in time, which comprises applying on the plant, part of the plant, or the surrounding area thereof, a pesticidal combination comprising, for example, at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activators), provided that a) if component (II) is acibenzolar-S-methyl then component (I) is not solely thiamethoxam, b) if component (II) is acibenzolar-S-methyl or harpin then component (I) is not solely abamectin or a mixture of abamectin with one of clothianidin, imidacloprid or thiamethoxam, c) if component (U) is acibenzolar-S-methyl or harpin then component (I) is not solely thiodicarb or a mixture of thiodicarb with one of clothianidin, imidacloprid or thiamethoxam, and d) if component (II) is acibenzolar-S-methyl or harpin then no plant growth regulator selected from the group comprising paclobutrazol, cyproconazole, uniconazole, tetcyclacis, trinexapac, gibberellic acid and gibberellic acid-3 is present.

The present invention relates to the use of a defined combination of pesticidal active ingredients, and compositions thereof, and methods for using such combinations in the control or prevention of pest and/or pathogenic damage, in particular in a plant propagation material and plant organs that grow at a later point in time by applying the combination on to the plant propagation material. The pests to be controlled in the present invention are insects, nematodes, acarines and molluscs, particularly insects and nematodes.

Pests, such as insects and nematodes, damage crops by direct feeding damage, by transmitting viruses and by facilitating bacterial and fungal infections. The damage caused by pests, such as insects and nematodes, to crops is often unspecific and easily confused with drought, malnutrition or disease. Typical symptoms are wilting, yellowing of the foliage and uneven or stunted growth.

Methods to control insects and thereby protect the plant include (1) use of insecticides (e.g. thiodicarb, clothianidin, imidacloprid) to control or eradicate insects usually by spraying or baiting, (2) use of biological control (e.g. insects, such as Solenopsis invicta, or nematodes, such as Neoaplectana carpocapsae) to control or eradicate insects, (3) use of crop rotation practices, which is effective against insects that are specific to a particular crop; however, insects that have different hosts cannot be controlled by this method, and (4) use of insect resistant or tolerant crops, which have been developed by conventional breeding or recombinant DNA technology (genetically modified plants).

Methods to control nematodes and thereby protect the plant include (1) use of nematicides (e.g., aldicarb) and fumigants (e.g., methyl bromide), (2) use of soil steaming, (3) use of crop rotation practices, which is effective against nematodes that are specific to a particular crop; however, nematodes that have different hosts cannot be controlled by this method, and (4) use of nematode resistant or tolerant crops, which have been developed by conventional breeding or recombinant DNA technology (genetically modified plants).

Certain combinations of active ingredients for controlling pests, such as insects and nematodes, and/or pathogenic damage are described in the literature. The biological properties of those known combinations are not entirely satisfactory in the areas of pathogenic control, phytotoxicity, and environmental and worker exposure, for example. In particular, in the instance a pathogen has become, or risks becoming resistant to the previously known combinations, improved methods of control or prevention are sought.

There is a continuing need to provide pesticidal combinations, which provide improved, for example, biological properties, for example, synergistic properties, especially for controlling pests, such as insects and nematodes, and/or pathogenic damage.

That need is solved according to the invention by the provision of the present pesticidal combination. Accordingly, in a first aspect, the present invention provides a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil, and abamectin and component (II) is one or more plant activator(s), provided that if component (II) is acibenzolar-S-methyl then component (I) is not solely thiamethoxam.

The term “insecticide” refers to a compound having an effect on, such as reduction in the damage caused by, agricultural-related insects. Non-limiting examples of insecticides include oxime carbamate insecticides (e.g., thiodicarb), neonicotinoid insecticides (e.g., clothianidin, imidacloprid, thiamethoxam), pyridylmethylamine insecticides (e.g., acetamiprid, thiacloprid), diamide insecticides (e.g., chlorantraniliprole), pyrethroid ester insecticides (e.g., beta-cyfluthrin, lambda-cyhalothrin, tefluthrin) and phenylpyrazole insecticides (e.g., fipronil), and avermectin insecticides (e.g., abamectin). Particularly preferred insecticides include thiodicarb, clothianidin, imidacloprid, thiamethoxam and fipronil. In some instances, thiodicarb is known to have nematicidal and/or molluscicidal characteristics and accordingly in such instances, the present invention also relates to combinations which contain thiodicarb in such instances. In some instances, fipronil and abamectin are known to have acaricidal characteristics and accordingly in such instances, the present invention also relates to combinations which contain fipronil or abamectin in such instances.

The term “plant activator” refers to a compound which does not act directly on the disease organism, nor does it alter the DNA of treated plants, but instead activates a natural defence mechanism in the host plant, referred to as systemic acquired resistance (SAR). Non-limiting examples of plant activators include acibenzolar-S-methyl, CGA 210007, benzoic acid, harpin, magnesium-dihydrojasmonate and salicylic acid. Particularly preferred plant activators include acibenzolar-S-methyl and harpin. In some instances, acibenzolar-S-methyl is known to have fungicidal and/or nematicidal characteristics and accordingly in such instances, the present invention also relates to combinations which contain acibenzolar-S-methyl in such instances. In some instances, harpin is known to have nematicidal characteristics and accordingly in such instances, the present invention also relates to combinations which contain harpin in such instances.

Each of the combinations demonstrates unexpected, for example synergistic activity, compared to activity of compounds alone.

In a second aspect, the present invention provides a method of controlling or preventing pest and/or pathogenic damage in a plant propagation material, a plant, and/or plant organs that grow at a later point in time, which comprises applying to the plant, part of the plant, or the surrounding area thereof the combination, as defined in the first aspect, in any desired sequence or simultaneously.

In a third aspect, the present invention provides a method of protecting a plant propagation material, a plant, and/or plant organs that grow at a later point in time against pest and/or pathogenic damage, which comprises applying to the plant, part of the plant, or the surrounding area thereof the combination, as defined in the first aspect, in any desired sequence or simultaneously.

In a fourth aspect, the present invention provides a method which comprises (i) treating a plant propagation material, such as a seed, with a pesticidal combination as defined in the first aspect, and (ii) planting or sowing the treated propagation material, wherein the combination protects against pest and/or pathogenic damage of the treated plant propagation material, parts of plant and/or plant grown from the treated propagation material.

In a fifth aspect, the present invention provides a method which comprises (i) treating a plant propagation material, such as a seed, with a pesticidal combination as defined in the first aspect, and (ii) planting or sowing the treated propagation material, and (iii) achieving protection against pest and/or pathogenic damage of the treated plant propagation material, parts of plant and/or plant grown from the treated propagation material.

In a sixth aspect, the present invention provides a method as defined above, wherein the active ingredient components of the combination, as defined in first aspect, are applied simultaneously.

In a seventh aspect, the present invention provides a method as defined above, wherein the combination, as defined in the first aspect, is applied on plant propagation material.

In an eighth aspect, the present invention relates to a plant propagation material treated with the combination as defined in the first aspect.

The components (I) and (II) defined in the first aspect are active ingredients for use in the agrochemical industry (also known as pesticides). A description of their structure as well as other pesticides (e.g., fungicides) can be found in The e-Pesticide Manual, Version 3.2, 13^{th} Edition, Editor C. D. S. Tomlin, British Crop Protection Council, 2005-06, with the exception of chlorantraniliprole, CGA 210007, benzoic acid, salicylic acid, harpin and magnesium-dihydrojasmonate for which alternative references have been provided.

Chlorantraniliprole (CAS RN 500008-45-7) was disclosed as an insecticide, for example, in WO 03/015518.

CGA 210007 (CAS RN 35272-27-6) was disclosed as a plant activator, for example, in European Journal of Plant Pathology (2002), 108(1), 41-49.

Benzoic acid (CAS RN 65-85-0) and salicylic acid (CAS RN 69-72-7) were disclosed as plant activators, for example, in EP 1,036,499.

Harpin (CAS RN 151438-54-9) was disclosed as a plant activator, for example, in WO 95/31564.

Magnesium-dihydrojasmonate was disclosed as a plant activator, for example, in Agrow Magazine (2006), 5, 23.

Controlling, preventing or protecting and its inflections, within the context of the present invention, mean reducing any undesired effect, such as
pest damage on, and
pathogenic, such as phytopathogenic, especially fungi, infestation or attack of, a plant, part of the plant or plant propagation material to such a level that an improvement is demonstrated.

The pesticidal combinations according to the invention have very advantageous properties for protecting plants against (i) pest attack or damage and/or (ii) pathogenic, such as phytopathogenic, especially fungi, attack or infestation, which result in a disease and damage to the plant; particularly in instance of plants, the present invention can control or prevent pest and/or pathogenic damage on a seed, parts of plant and/or plant grown from the treated seed. In some cases, control against pest attack or damage also indirectly results in control against pathogenic attack, and vice-a-versa.

These properties are, for example, the synergistically enhanced action of combinations of components (I) and (II), resulting in lower pest and/or pathogenic damage, lower rates of application, or a longer duration of action. In the instance of agriculture, the enhanced action is found to show an improvement in the growing characteristics of a plant by, for example, higher than expected control of pest and/or pathogenic damage.

The improvement in the growing (or growth) characteristics of a plant can manifest in a number of different ways, but ultimately it results in a better product of the plant. It can, for example, manifest in improving the yield and/or vigour of the plant or quality of the harvested product from the plant, which improvement may not be not connected to the control of pests.

As used herein the phrase “improving the yield” of a plant relates to an increase in the yield of a product of the plant by a measurable amount over the yield of the same product of the plant produced under the same conditions, but without the application of the subject method. It is preferred that the yield be increased by at least about 0.5%, more preferred that the increase be at least about 1%, even more preferred is about 2%, and yet more preferred is about 4%, or more. Yield can be expressed in terms of an amount by weight or volume of a product of the plant on some basis. The basis can be expressed in terms of time, growing area, weight of plants produced, amount of a raw material used, or the like.

As used herein the phrase “improving the vigour” of a plant relates to an increase or improvement of the vigour rating, or the stand (the number of plants per unit of area), or the plant height, or the plant canopy, or the visual appearance (such as greener leaf colour), or the root rating, or emergence, or protein content, or increased tillering, or bigger leaf blade, or less dead basal leaves, or stronger tillers, or less fertilizer needed, or less seeds needed, or more productive tillers, or earlier flowering, or early grain maturity, or less plant verse (lodging), or increased shoot growth, or earlier germination, or any combination of these factors, or any other advantages familiar to a person skilled in the art, by a measurable or noticeable amount over the same factor of the plant produced under the same conditions, but without the application of the subject method. When it is said that the present method is capable of “improving the yield and/or vigour” of a plant, the present method results in an increase in either the yield, as described above, or the vigour of the plant, as described above, or both the yield and the vigour of the plant.

Accordingly, the present invention also provides a method of improving the growing characteristics of a plant, and/or plant organs that grow at a later point in time, which comprises applying to the plant, part of the plant, or the surrounding area thereof the combination, as defined in the first aspect, in any desired sequence or simultaneously.

In a preferred embodiment of any aspect of the invention, each combination is a composition comprising, preferably of, components (I) and (II), and optionally one or more customary formulation auxiliaries.

In another preferred embodiment of any aspect of the invention, are combinations of the invention wherein component (I) is one insecticide.

In a further preferred embodiment of any aspect of the invention, are combinations of the invention wherein component (II) is one plant activator.

In yet a further preferred embodiment of any aspect of the invention, are combinations of the invention wherein component (I) is one insecticide and component (II) is one plant activator.

Preferred embodiments are combinations comprising
(a) thiodicarb as component (I) and acibenzolar-S-methyl as component (II);
(b) clothianidin as component (I) and acibenzolar-S-methyl as component (II);
(c) imidacloprid as component (I) and acibenzolar-S-methyl as component (II);
(d) acetamiprid as component (I) and acibenzolar-S-methyl as component (II);
(e) thiacloprid as component (I) and acibenzolar-S-methyl as component (II);
(f) chlorantraniliprole as component (I) and acibenzolar-S-methyl as component (II);
(g) beta-cyfluthrin as component (I) and acibenzolar-S-methyl as component (II);
(h) lambda-cyhalothrin as component (I) and acibenzolar-S-methyl as component (II);
(j) tefluthrin as component (I) and acibenzolar-S-methyl as component (II);
(k) fipronil as component (I) and acibenzolar-S-methyl as component (II);
(m) thiodicarb as component (I) and harpin as component (II);
(n) clothianidin as component (I) and harpin as component (II);
(o) imidacloprid as component (I) and harpin as component (II);
(p) acetamiprid as component (I) and harpin as component (II);
(q) thiacloprid as component (I) and harpin as component (II);
(r) chlorantraniliprole as component (I) and harpin as component (II);
(s) beta-cyfluthrin as component (I) and harpin as component (II);
(t) lambda-cyhalothrin as component (I) and harpin as component (II);
(u) tefluthrin as component (I) and harpin as component (II);
(v) fipronil as component (I) and harpin as component (II); and
(w) thiamethoxam as component (I) and harpin as component (II).

Each of the combinations of the invention can be used in the agricultural sector and related fields of use for controlling or preventing pest and/or pathogenic damage on plants.

Each of the combinations according to the present invention is effective against insects that can be inhibited using such a treatment regimen include, but are not limited to, pests selected from Insecta and Arachnida. In that instance, the combination can also be applied on the pest to control or prevent pest damage and protect the desired material (e.g., plant and parts of plant) from pest damage. Examples of pests include:
from the order Lepidoptera (e.g., Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia spp., Cryptophlebia leucotreta, Crysodeixis includens, Cydia spp., Diatraea spp., Diparopsis castanea, Earias sspp., Elasmopalpus spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiella, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichoplusia spp. and Yponomeuta spp.);
from the order Coleoptera (e.g., Agriotes spp., Anthonomus spp., Atomaria linearis, Ceutorhynchus spp., Chaetocnema tibialis, Cosmopolites spp., Curculio spp., Dermestes spp., Diabrotica spp., Epilachna spp., Eremnus spp., Gonocephalum spp., Heteronychus spp., Leptinotarsa decemlineata, Lissorhoptrus spp., Melolontha spp., Orycaephilus spp., Otiorhynchus spp., Phlyctinus spp., Phyllotreta spp., Popillia spp., Protostrophus spp., Psylliodes spp., Rhizopertha spp., Scarabeidae, Sitophilus spp., Sitotroga spp., Tenebrio spp., Tribolium spp. and Trogoderma spp.);
from the order Orthoptera (e.g., Blatta spp., Blattella spp., Gryllotalpa spp., Leucophaea maderae, Locusta spp., Periplaneta spp. and Schistocerca spp.);
from the order Isoptera (e.g., Reticulitermes spp.);
from the order Psocoptera (e.g., Liposcelis spp.);
from the order Anoplura (e.g., Haematopinus spp., Linognathus spp., Pediculus spp., Pemphigus spp. and Phylloxera spp.);
from the order Mallophaga (e.g., Damalinea spp. and Trichodectes spp.);
from the order Thysanoptera (e.g., Frankliniella spp., Hercinothrips spp., Taeniothrips spp., Thrips palmi, Thrips tabaci and Scirtothrips aurantii);
from the order Heteroptera (e.g., Dichelops melacanthus, Distantiella theobroma, Dysdercus spp., Euchistus spp., Eurygaster spp., Leptocorisa spp., Nezara spp., Piesma spp., Rhodnius spp., Sahlbergella singularis, Scotinophara spp. and Triatoma spp.);
from the order Homoptera (e.g., Aleurothrixus floccosus, Aleyrodes brassicae, Aonidiella spp., Aphididae spp., Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Paratoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri);
from the order Hymenoptera (e.g., Acromyrmex spp., Athalia rosae, Atta spp., Cephus spp., Diprion spp., Diprionidae spp., Gilpinia polytoma, Hoplocampa spp., Lasius spp., Monomorium pharaonis, Neodiprion spp., Solenopsis spp. and Vespa spp.);
from the order Diptera (e.g., Antherigona soccata, Bibio hortulanus, Ceratitis spp., Chrysomyia spp., Culex spp., Cuterebra spp., Dacus spp., Delia spp., Drosophila melanogaster, Liriomyza spp., Melanagromyza spp., Orseolia spp., Oscinella frit, Pegomyia hyoscyami, Phorbia spp., Rhagoletis pomonella, Sciara spp.); and
from the order Acarina (e.g., Acarus siro, Aceria sheldoni, Aculus schlechtendali, Amblyomma spp., Argas spp., Brevipalpus spp., Bryobia praetiosa, Calipitrimerus spp., Chorioptes spp., Dermanyssus gallinae, Eotetranychus carpini, Eriophyes spp., Hyalomma spp., Olygonychus pratensis, Ornithodoros spp., Panonychus spp., Phyllocoptruta oleivora, Polyphagotarsonemus latus, Psoroptes spp., Rhipicephalus spp., Rhizoglyphus spp., Sarcoptes spp., Tarsonemus spp. and Tetranychus spp.).

Each of the combinations according to the present invention is effective against nematodes that can be inhibited using such a treatment regimen include, but are not limited to, root knot nematodes, cyst-forming nematodes, stem eelworms and foliar nematodes. In particular, nematodes of the following species can be managed using the combination treatments of the invention: Anguina spp., Aphelenchoides spp., Belonolaimus spp., Criconemella spp., Criconemoides spp., Ditylenchus spp., Dolichodorus spp., Globodera spp. (e.g., Globodera rostochiensis), Helicotylenchus spp., Heterodera spp. (e.g., Heterodera schachtii, Heterodora avenae, Heterodera glycines, and Heterodora trifolii), Hemicriconemoides spp., Hemicycliophora spp., Hirschmaniella spp., Hoplolaimus spp., Hypsoperine spp., Longidorus spp., Macroposthonia spp., Melinius spp., Meloidogyne spp. (e.g., Meloidogyne incognita and Meloidogyne javanica), Nacobbus spp., Paratrichodorus spp., Pratylenchus spp. (e.g., Pratylenchus neglectans and Pratylenchus penetrans), Punctodera spp., Quinisulcius spp., Radopholus spp. (e.g., Radopholus similis), Rotylenchulus spp., Scutellonema spp., Subanguina spp., Trichodorus spp., Tylenchorhynchus spp., Tylenchulus spp. (e.g., Tylenchulus semipenetrans) and Xiphinema spp.

The combinations of the invention can be formulated for a particular use. Preferably, the combination is formulated for protecting cultivated plants or their propagation materials. Advantageously, the combinations are formulated for seed treatment applications for controlling or preventing damage by pests, which are found in agriculture and forestry, and can particularly damage the plant in the early stages of its development.

Further, the combinations of the invention can be applied to the plant and/or part of the plant, in a conventional manner, such as foliar spray. Thus the benefit from the invention can be achieved either by (a) treating plant propagation material with a combination or (b) applying to the plant and/or part of the plant developing from the plant propagation material a combination, or both (a) and (b).

Further, the present invention also envisages soil application of the combinations of the invention to control the soil-dwelling pests and/or soil-borne pathogens. Methods of applying to the soil can be via any suitable method, which ensures that the combination penetrates the soil, for example, nursery tray application, in furrow application, soil drenching, soil injection, drip irrigation, application through sprinklers or central pivot, incorporation into soil (broad cast or in band) are such methods. Thus the benefit from the invention can be achieved either by (A) treating plant propagation material with a combination or (B) applying to the locus where control is desired, generally the planting site, a combination, or both (A) and (B). For example, a combination of the invention can be applied to bare soil, that is in the absence of plant or plant propagation material, and the plant propagation material can then advantageously be planted into such treated soil. Alternatively, the combination of the invention can be applied to the soil at the same time as the plant propagation material is planted into said soil, for example by sprinkling in granules of the composition. Another possibility is that the plant propagation material is planted into untreated soil and the combination of the invention is then applied to the locus of the planted plant propagation material, for example by drenching with a solution of the composition. Likewise, the benefit from the invention can be achieved either by (A) applying to the locus where control is desired, generally the planting site, a combination or (B) applying to the plant or parts of the plant developing from the plant propagation material a combination, or both (A) and (B).

The term “plant propagation material” is understood to denote all the generative parts of the plant, such as seeds, which can be used for the multiplication of the latter and vegetative plant material, such as cuttings and tubers (for example, potatoes). Accordingly, as used herein, part of a plant includes propagation material. There may be mentioned, e.g., the seeds (in the strict sense), roots, fruits, tubers, bulbs, rhizomes, parts of plants. Germinated plants and young plants, which are to be transplanted after germination or after emergence from the soil, may also be mentioned. These young plants may be protected before transplantation by a total or partial treatment by immersion.

Parts of plant and plant organs that grow at later point in time are any sections of a plant that develop from a plant propagation material, such as a seed. Parts of plant, plant organs, and plants can also benefit from the pest and/or pathogenic damage protection achieved by the application of the combination on to the plant propagation material. In an embodiment, certain parts of plant and certain plant organs that grow at later point in time can also be considered as plant propagation material, which can themselves be applied (or treated) with the combination; and consequently, the plant, further parts of the plant and further plant organs that develop from the treated parts of plant and treated plant organs can also benefit from the pest and/or pathogenic damage protection achieved by the application of the combination on to the certain parts of plant and certain plant organs.

Methods for applying or treating pesticidal active ingredients and mixtures thereof on to plant propagation material, especially seeds, are known in the art, and include dressing, coating, pelleting and soaking application methods of the propagation material.

The active ingredients can be applied to the seeds using conventional treating techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art.

In a preferred embodiment, the combination is applied or treated on to the plant propagation material by a method such that the germination is not induced; generally seed soaking induces germination because the moisture content of the resulting seed is too high. Accordingly, preferred examples of suitable methods for applying (or treating) a plant propagation material, such as a seed, is seed dressing, seed coating or seed pelleting and alike.

It is preferred that the plant propagation material is a seed. Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed either before or after the treatment or the treatment may be applied during the priming process.

Even distribution of the active ingredients and adherence thereof to the seeds is desired during propagation material treatment. Treatment could vary from a thin film (dressing) of the formulation containing the active ingredient(s) on a plant propagation material, such as a seed, where the original size and/or shape are recognizable to an intermediary state (such as a coating) and then to a thicker film (such as pelleting with many layers of different materials (such as carriers, for example, clays; different formulations, such as of other active ingredients; polymers; and colourants) where the original shape and/or size of the seed is no longer recognisable.

The seed treatment occurs to an unsown seed, and the term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil but would include any application practice that would target the seed during the planting process.

Preferably, the treatment occurs before sowing of the seed so that the sown seed has been pre-treated with the combination. In particular, seed coating or seed pelleting are preferred in the treatment of the combinations according to the invention. As a result of the treatment, the active ingredients in the combination are adhered on to the seed and therefore available for pest control.

The treated seeds can be stored, handled, sowed and tilled in the same manner as any other active ingredient treated seed.

The combination according to the present invention is suitable for plants of the crops: cereals (wheat, barley, rye, oats, maize, rice, sorghum, triticale and related crops); beet (sugar beet and fodder beet); leguminous plants (beans, lentils, peas, soybeans); oil plants (rape, mustard, canola, sunflowers); cucumber plants (marrows, cucumbers, melons); fibre plants (cotton, flax, hemp, jute); vegetables (spinach, lettuce, asparagus, cabbages, broccoli, cauliflower, carrots, onions, tomatoes, peppers, potatoes, paprika); as well as ornamentals (flowers, shrubs, broad-leaved trees and evergreens, such as conifers). Especially suitable are cotton, maize, soybeans, sugar beet, cucumber plants and vegetables.

Suitable target crops also include transgenic crop plants of the foregoing types. The transgenic crop plants used according to the invention are plants, or propagation material thereof, which are transformed by means of recombinant DNA technology in such a way that they are—for instance—capable of synthesizing selectively acting toxins as are known, for example, from toxin-producing invertebrates, especially of the phylum Arthropoda, as can be obtained from Bacillus thuringiensis strains; or as are known from plants, such as lectins; or in the alternative capable of expressing a herbicidal or fungicidal resistance. Examples of such toxins, or transgenic plants which are capable of synthesizing such toxins, have been disclosed, for example, in EP-A-0 374 753, WO 93/07278, WO 95/34656, EP-A-0 427 529 and EP-A-451 878 and are incorporated by reference in the present application.

The plant propagation material treated by a combination of the present invention are, therefore, resistant to pest and/or pathogenic damage; accordingly, the present invention also provides pest resistant plant propagation material which is treated with the combination and consequently at least the active ingredients thereof are adhered on the propagation material, such a seed.

The seed treatment combination and composition can also comprise or may be applied together and/or sequentially with further active compounds. These further compounds can be other pesticidal active ingredients, fertilizers, antioxidants or micronutrient donors or other preparations that influence plant growth, such as inoculants.

A single pesticidal active ingredient may have activity in more than area of disease or pest control, for example, a pesticide may have fungicide, insecticide and nematicide activity. Specifically, thiodicarb is known for insecticide, nematicide and molluscicide activity, while fipronil and abamectin are known for insecticide and acaricide activity, and acibenzolar-S-methyl is known as a plant activator and for fungicide and/or nematicide activity. Furthermore, harpin is known as a plant activator and for nematicide activity.

The combination of the present invention may be mixed with other pesticides, such as further insecticides or fungicides.

Suitable examples of further insecticides for appropriate combinations of the invention include further neonicotinoid insecticides (e.g., thiamethoxam) and avermectin insecticides (e.g., abamectin), and mixtures thereof.

In a preferred embodiment, combinations (a) to (v) specifically further comprises one or more of (a) thiamethoxam and/or (b) abamectin.

Suitable non-limiting examples of fungicides include phenylpyrrole fungicides (e.g., fludioxonil), phenylamide fungicides (e.g., metalaxyl, mefenoxam), strobilurin fungicides (e.g., azoxystrobin), and triazole fungicides (e.g., difenoconazole, myclobutanil), and mixtures thereof.

In a preferred embodiment, the combination further comprises one or more of (a) fludioxonil, (b) metalaxyl, (c) mefenoxam, (d) azoxystrobin, (e) difenoconazole and/or (f) myclobutanil.

The combinations according to the present invention when mixed with fungicides are also effective against phytopathogenic fungi, especially occurring in plants, including seed-borne fungi and belong to the following classes: Ascomycetes (e.g., Penicillium, Gaeumannomyces graminis); Basidiomycetes (e.g., the genus Hemileia, Rhizoctonia, Puccinia); Fungi imperfecti (e.g., Botrytis, Helminthosporium, Rhynchosporium, Fusarium, Septoria, Cercospora, Alternaria, Pyricularia, Thelaviopsis and Pseudocercosporella herpotrichoides); Oomycetes (e.g., Phytophthora, Peronospora, Bremia, Pythium, Plasmopara and Aphanomyces); Zygomycetes (e.g., Rhizopus spp.). A combination is especially effective against Alternaria spp., Aspergillus spp., Claviceps purpurea, Cochliobolus spp., Colletotrichum spp., Diplodia maydis, Erysiphe graminis, Fusarium spp. (e.g., Fusarium culmorum, Fusarium oxysporium, Fusarium solani, Fusarium graminearum and Fusarium moniliforme), Gaeumannomyces graminis, Giberella fujikuroi, Giberella zeae, Helminthosporium graminearum, Monographella nivalis, Puccinia spp., Pyrenophora spp. (e.g., Pyrenophora graminea), Peronosclerospora spp., Peronspora spp., Phakopsora pachyrhizi, Phythium spp., Phoma spp., Phomopsis spp., Rhizoctonia solani, Septoria spp., Pseudocercosporella spp., Thelaviopsis spp., Tilletia spp., Rhizopus spp., Typhula spp., Ustilago spp., Sphacelotheca spp. (e.g., Spacelotheca reilliani), Thanatephorus cucumeris, and Verticillium spp.

The weight ratio of active ingredients in the combination vary, for example, according to type of use, type of crop, the specific active ingredients in the combination, type of plant propagation material (if appropriate), but is such that the active ingredients in the combination are in an effective proportion to provide the desired enhanced action (such as pest and/or pathogenic control) and can be determined by trials known to one of ordinary skill in the art.

The weight ratio of active ingredient compounds is selected as to give the desired, for example synergistic, action. In general, the weight ratio would vary depending on the specific active ingredient and how many active ingredients are present in the combination. Generally, in the event the combination consists of two components (I) and (II) the weight ratio between components (I) and (II) is from 10000:1 to 1:1000, more preferably from 1000:1 to 1:100, most preferably from 100:1 to 1:10.

The rates of application (use) of the combination vary, for example, according to type of use, type of crop, the specific active ingredients in the combination, type of plant propagation material (if appropriate), but is such that the active ingredients in the combination are in an effective amount to provide the desired enhanced action (such as pest and/or pathogenic control) and can be determined by trials known to one of ordinary skill in the art.

Generally for seed treatment, application rates can vary from 0.1 μg to 10 mg of active ingredients per seed. Examples of application rates for seed treatment tend to be 0.01 mg-10 mg, preferably 0.1 mg-1.0 mg of component (I) per seed; and 0.1 μg-10 mg, and preferably 1.0 μg-1.0 mg of component (II) per seed.

In the event the combination comprises (a) thiodicarb as component (I) and acibenzolar-S-methyl as component (II), typical application rates for seed treatment, is 300-1000 g of thiodicarb per 100 kg seed, and 1.0-500 μg of acibenzolar-S-methyl per seed.

In the event the combination comprises (b) clothianidin as component (I) and acibenzolar-S-methyl as component (II), typical application rates for seed treatment, is 25-600 g of clothianidin per 100 kg seed, and 1.0-500 μg of acibenzolar-S-methyl per seed.

In the event the combination comprises (c) imidacloprid as component (I) and acibenzolar-S-methyl as component (II), typical application rates for seed treatment, is 25-600 g of imidacloprid per 100 kg seed, and 1.0-500 μg of acibenzolar-S-methyl per seed.

In the event the combination comprises (d) acetamiprid as component (I) and acibenzolar-S-methyl as component (II), typical application rates for seed treatment, is 50-300 g of acetamiprid per 100 kg seed, and 1.0-500 μg of acibenzolar-S-methyl per seed.

In the event the combination comprises (e) thiacloprid as component (I) and acibenzolar-S-methyl as component (II), typical application rates for seed treatment, is 50-300 g of thiacloprid per 100 kg seed, and 1.0-500 μg of acibenzolar-S-methyl per seed.

In the event the combination comprises (f) chlorantraniliprole as component (I) and acibenzolar-S-methyl as component (II), typical application rates for seed treatment, is 0.01-1.0 mg of chlorantraniliprole per seed, and 1.0-500 μg of acibenzolar-S-methyl per seed.

In the event the combination comprises (g) beta-cyfluthrin as component (I) and acibenzolar-S-methyl as component (II), typical application rates for seed treatment, is 12.5-60 g of beta-cyfluthrin per 100 kg seed, and 1.0-500 μg of acibenzolar-S-methyl per seed.

In the event the combination comprises (h) lambda-cyhalothrin as component (I) and acibenzolar-S-methyl as component (II), typical application rates for seed treatment, is 12.5-60 g of lambda-cyhalothrin per 100 kg seed, and 1.0-500 μg of acibenzolar-S-methyl per seed.

In the event the combination comprises (j) tefluthrin as component (I) and acibenzolar-S-methyl as component (II), typical application rates for seed treatment, is 12.5-60 g of tefluthrin per 100 kg seed, and 1.0-500 μg of acibenzolar-S-methyl per seed.

In the event the combination comprises (k) fipronil as component (I) and acibenzolar-S-methyl as component (II), typical application rates for seed treatment, is 0.125-1.25 mg of fipronil per seed, and 1.0-500 μg of acibenzolar-S-methyl per seed.

In the event the combination comprises (m) thiodicarb as component (I) and harpin as component (II), typical application rates for seed treatment, is 300-1000 g of thiodicarb per 100 kg seed, and 1.0-10 g of harpin per 100 kg seed.

In the event the combination comprises (n) clothianidin as component (I) and harpin as component (II), typical application rates for seed treatment, is 25-600 g of clothianidin per 100 kg seed, and 1.0-10 g of harpin per 100 kg seed.

In the event the combination comprises (o) imidacloprid as component (I) and harpin as component (II), typical application rates for seed treatment, is 25-600 g of imidacloprid per 100 kg seed, and 1.0-10 g of harpin per 100 kg seed.

In the event the combination comprises (p) acetamiprid as component (I) and harpin as component (II), typical application rates for seed treatment, is 50-300 g of acetamiprid per 100 kg seed, and 1.0-10 g of harpin per 100 kg seed.

In the event the combination comprises (q) thiacloprid as component (I) and harpin as component (II), typical application rates for seed treatment, is 50-300 g of thiacloprid per 100 kg seed, and 1.0-10 g of harpin per 100 kg seed.

In the event the combination comprises (r) chlorantraniliprole as component (I) and harpin as component (II), typical application rates for seed treatment, is 0.01-1.0 mg of chlorantraniliprole per seed, and 1.0-10 g of harpin per 100 kg seed.

In the event the combination comprises (s) beta-cyfluthrin as component (I) and harpin as component (II), typical application rates for seed treatment, is 12.5-60 g of beta-cyfluthrin per 100 kg seed, and 1.0-10 g of harpin per 100 kg seed.

In the event the combination comprises (t) lambda-cyhalothrin as component (I) and harpin as component (II), typical application rates for seed treatment, is 12.5-60 g of lambda-cyhalothrin per 100 kg seed, and 1.0-10 g of harpin per 100 kg seed.

In the event the combination comprises (u) tefluthrin as component (I) and harpin as component (II), typical application rates for seed treatment, is 12.5-60 g of tefluthrin per 100 kg seed, and 1.0-10 g of harpin per 100 kg seed.

In the event the combination comprises (v) fipronil as component (I) and harpin as component (II), typical application rates for seed treatment, is 0.125-1.25 mg of fipronil per seed, and 1.0-10 g of harpin per 100 kg seed.

In the event the combination comprises (w) thiamethoxam as component (I) and harpin as component (II), typical application rates for seed treatment, is 25-600 g of thiamethoxam per 100 kg seed, and 1.0-10 g of harpin per 100 kg seed.

The components (I) or (II), and any other additional pesticides, may be used either in pure form, i.e., as a solid active ingredient, for example, in a specific particle size, or preferably together with at least one of the auxiliaries (also known as adjuvants) customary in formulation technology, such as extenders, e.g., solvents or solid carriers, or surface-active compounds (surfactants), in the form of a formulation, in the present invention. Generally, the compounds (I) and (II) are in the form of a formulation composition with one or more of customary formulation auxiliaries.

Therefore, the combination of compounds (I) and (II) are normally used in the form of formulations. The compounds (I) and (II) can be applied to the locus where control is desired either simultaneously or in succession at short interval, for example on the same day, if desired together with further carriers, surfactants or other application-promoting adjuvants customarily employed in formulation technology. In a preferred embodiment, (I) and (II) are applied simultaneously.

In the event compounds (I) and (II) are applied simultaneously in the present invention, they may be applied as a composition containing (I) and (II), in which case each of (I) and (II) can be obtained from a separate formulation source and mixed together (known as a tank-mix, ready-to-apply, spray broth, or slurry), optionally with other pesticides, or (I) and (II) can be obtained as single formulation mixture source (known as a pre-mix, concentrate, formulated compound (or product)), and optionally mixed together with other pesticides.

In an embodiment, the combination of the present invention is applied as a composition. Accordingly, the present invention is a composition comprising, as active ingredients, (I) and (II), and optionally other pesticides, and optionally one or more customary formulation auxiliaries; which may be in the form of a tank-mix or pre-mix composition.

In a preferred embodiment, each of the combinations of the invention are provided in the form of a pre-mix composition (or mixture): suitable examples being combinations of
(a) thiodicarb as component (I) and acibenzolar-S-methyl as component (II);
(b) clothianidin as component (I) and acibenzolar-S-methyl as component (II);
(c) imidacloprid as component (I) and acibenzolar-S-methyl as component (II);
(k) fipronil as component (I) and acibenzolar-S-methyl as component (II);
(m) thiodicarb as component (I) and harpin as component (II);
(n) clothianidin as component (I) and harpin as component (II);
(o) imidacloprid as component (I) and harpin as component (II);
(v) fipronil as component (I) and harpin as component (II); and
(w) thiamethoxam as component (I) and harpin as component (II).

Examples of foliar formulation types for pre-mix compositions are:
GR: Granules (mainly used for direct soil treatment)
WP: wettable powders
WG: water dispersible granules (powders)
SG: water soluble granules
SL: soluble concentrates
EC: emulsifiable concentrate
EW: emulsions, oil in water
ME: micro-emulsion
SC: aqueous suspension concentrate
CS: aqueous capsule suspension
OD: oil-based suspension concentrate, and
SE: aqueous suspo-emulsion.

Whereas, examples of seed treatment formulation types for pre-mix compositions are:
WS: wettable powders for seed treatment slurry
LS: solution for seed treatment
ES: emulsions for seed treatment
FS: suspension concentrate for seed treatment
WG: water dispersible granules, and
CS: aqueous capsule suspension.

Examples of formulation types suitable for tank-mix compositions are solutions, dilute emulsions, suspensions, or a mixture thereof, and dusts.

As with the nature of the formulations, the methods of application, such as foliar, drench, spraying, atomizing, dusting, scattering, coating or pouring, are chosen in accordance with the intended objectives and the prevailing circumstances.

The tank-mix compositions are generally prepared by diluting with a solvent (for example, water) the one or more pre-mix compositions containing different pesticides, and optionally further auxiliaries.

Suitable carriers and adjuvants can be solid or liquid and are the substances ordinarily employed in formulation technology, e.g. natural or regenerated mineral substances, solvents, dispersants, wetting agents, tackifiers, thickeners, binders or fertilizers.

The formulations are prepared in known manner, e.g., by homogeneously mixing and/or grinding the active ingredients with extenders, e.g., solvents, solid carriers and, where appropriate, surface-active compounds (surfactants).

Suitable solvents include: aromatic hydrocarbons, preferably the fractions containing 8 to 12 carbon atoms, e.g. xylene mixtures or substituted naphthalenes, phthalates, such as dibutyl phthalate or dioctyl phthalate, aliphatic hydrocarbons, such as cyclohexane or paraffins, alcohols and glycols and their ethers and esters, such as ethanol, ethylene glycol, ethylene glycol monomethyl or monoethyl ether, ketones, such as cyclohexanone, strongly polar solvents, such as N-methyl-2-pyrrolidone, dimethyl sulfoxide or dimethylformamide, as well as vegetable oils or epoxidised vegetable oils, such as epoxidised coconut oil or soybean oil; or water.

The solid carriers used, e.g., for dusts and dispersible powders, are normally natural mineral fillers, such as calcite, talcum, kaolin, montmorillonite or attapulgite. In order to improve the physical properties it is also possible to add highly dispersed silicic acid or highly dispersed absorbent polymers. Suitable granulated adsorptive carriers include porous types, for example pumice, broken brick, sepiolite or bentonite, and suitable nonsorbent carriers are, for example, calcite or sand. In addition, a great number of pregranulated materials of inorganic or organic nature can be used, e.g., especially dolomite or pulverized plant residues.

Depending upon the nature of the active ingredient compounds to be formulated, suitable surface-active compounds are non-ionic, cationic and/or anionic surfactants having good emulsifying, dispersing and wetting properties. The term “surfactants” will also be understood as comprising mixtures of surfactants.

Particularly advantageous application-promoting adjuvants are also natural or synthetic phospholipids of the cephalin and lecithin series, e.g., phosphatidylethanolamine, phosphatidylserine, phosphatidylglycerol and lysolecithin.

Generally, a tank-mix formulation for foliar or soil application comprises 0.1 to 20%, especially 0.1 to 15%, active ingredient compounds, and 99.9 to 80%, especially 99.9 to 85%, of a solid or liquid auxiliaries (including, for example, a solvent such as water), where the auxiliaries can be a surfactant in an amount of 0 to 20%, especially 0.1 to 15%, based on the tank-mix formulation.

Typically, a pre-mix formulation for foliar application comprises 0.1 to 99.9%, especially 1 to 95%, active ingredient compounds, and 99.9 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Normally, a tank-mix formulation for seed treatment application comprises 0.25 to 80%, especially 1 to 75%, active ingredient compounds, and 99.75 to 20%, especially 99 to 25%, of a solid or liquid auxiliaries (including, for example, a solvent such as water), where the auxiliaries can be a surfactant in an amount of 0 to 40%, especially 0.5 to 30%, based on the tank-mix formulation.

Typically, a pre-mix formulation for seed treatment application comprises 0.5 to 99.9%, especially 1 to 95%, active ingredient compounds, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Whereas commercial products will preferably be formulated as concentrates (e.g., pre-mix composition (formulation)), the end user will normally employ dilute formulations (e.g., tank mix composition).

Preferred seed treatment pre-mix formulations are aqueous suspension concentrates. The formulation can be applied to the seeds using conventional treating techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art.

The Examples which follow serve to illustrate the formulations suitable for compounds (I) and (II), “active ingredient” denoting a combination of compound (I) and compound (II) in a specific mixing ratio.

### FORMULATION EXAMPLES

| Wettable powders | a) | b) | c) |
|---|---|---|---|
| active ingredient [I:II = 1:6(a), 1:2(b), 1:1(c)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders which can be diluted with water to give suspensions of the desired concentration.

| Dusts | a) | b) | c) |
|---|---|---|---|
| active ingredient [I:II = 1:6(a), 1:2(b), 1:10(c)] | 5% | 6% | 4% |
| Talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can be used for dry dressings for seed.

| Suspension concentrates | (a) | (b) |
|---|---|---|
| active ingredient (I:II = 1:1(a); 1:8(b)) | 5% | 30% |
| propylene glycol | 10% | 10% |
| Tristyrylphenol ethoxylates | 5% | 6% |
| sodium lignosulfonate | — | 10% |
| carboxymethylcellulose | — | 1% |
| silicone oil (in the form of a 75% emulsion in water) | 1% | 1% |
| Colour pigment | 5% | 5% |
| water | 74% | 37% |

The finely ground active ingredient is intimately mixed with the adjuvants, giving a suspension concentrate from which suspensions of any desired dilution can be obtained by dilution with water. Alternatively, a suspension of the active ingredients and auxiliaries (including water) is wet milled with a bead-mill to achieve a stable formulation and with the appropriate treatment characteristics.

Using such dilutions plant propagation material can be treated and protected against damage, for example, from pathogen(s), by spraying, pouring or immersing.

The active ingredient combinations according to the invention are distinguished by the fact that they are especially well tolerated by plants and are environmentally friendly.

Each active ingredient combination according to the invention is especially advantageous for the treatment of plant propagation material.

In a preferred embodiment, each of the combinations of the present invention is a plant propagation material, preferably seed, treating composition.

In one embodiment the invention relates to a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) if component (II) is acibenzolar-S-methyl or harpin then component (I) is not solely abamectin or a mixture of abamectin with one of clothianidin, imidacloprid or thiamethoxam,
c) if component (II) is acibenzolar-S-methyl or harpin then component (I) is not solely thiodicarb or a mixture of thiodicarb with one of clothianidin, imidacloprid or thiamethoxam, and
d) if component (II) is acibenzolar-S-methyl or harpin then no plant growth regulator selected from the group comprising paclobutrazol, cyproconazole, uniconazole, tetcyclacis, trinexapac, gibberellic acid and gibberellic acid-3 is present.

In another embodiment the invention relates to a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) component (I) is not solely abamectin or a mixture of abamectin with one of clothianidin, imidacloprid or thiamethoxam,
c) component (I) is not solely thiodicarb or a mixture of thiodicarb with one of clothianidin, imidacloprid or thiamethoxam, and
d) no plant growth regulator selected from the group comprising paclobutrazol, cyproconazole, uniconazole, tetcyclacis, trinexapac, gibberellic acid and gibberellic acid-3 is present.

In a further embodiment the invention relates to a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) component (I) is not solely abamectin or a mixture of abamectin with one of clothianidin, imidacloprid or thiamethoxam,
c) component (I) is not solely thiodicarb or a mixture of thiodicarb with one of clothianidin, imidacloprid or thiamethoxam, and
d) no plant growth regulator is present.

In one embodiment the invention relates to a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) if component (II) is acibenzolar-S-methyl or harpin then component (I) is not solely imidacloprid,
c) if component (II) is acibenzolar-S-methyl or harpin then component (I) is not solely abamectin or a mixture of abamectin with one of clothianidin, imidacloprid or thiamethoxam,
d) if component (II) is acibenzolar-S-methyl or harpin then component (I) is not solely thiodicarb or a mixture of thiodicarb with one of clothianidin, imidacloprid or thiamethoxam, and
e) if component (II) is acibenzolar-S-methyl or harpin then no plant growth regulator selected from the group comprising paclobutrazol, cyproconazole, uniconazole, tetcyclacis, trinexapac, gibberellic acid and gibberellic acid-3 is present.

In another embodiment the invention relates to a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) if component (II) is acibenzolar-S-methyl or harpin then component (I) is not solely imidacloprid,
c) component (I) is not solely abamectin or a mixture of abamectin with one of clothianidin, imidacloprid or thiamethoxam,
d) component (I) is not solely thiodicarb or a mixture of thiodicarb with one of clothianidin, imidacloprid or thiamethoxam, and
e) no plant growth regulator selected from the group comprising paclobutrazol, cyproconazole, uniconazole, tetcyclacis, trinexapac, gibberellic acid and gibberellic acid-3 is present.

In a further embodiment the invention relates to a pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) if component (II) is acibenzolar-S-methyl or harpin then component (I) is not solely imidacloprid,
c) component (I) is not solely abamectin or a mixture of abamectin with one of clothianidin, imidacloprid or thiamethoxam,
d) component (I) is not solely thiodicarb or a mixture of thiodicarb with one of clothianidin, imidacloprid or thiamethoxam, and
e) no plant growth regulator is present.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

1. A pesticidal combination comprising at least two active ingredient components optionally together with one or more customary formulation auxiliaries, wherein component (I) is one or more insecticide(s) selected from the group comprising thiodicarb, clothianidin, imidacloprid, thiamethoxam, acetamiprid, thiacloprid, chlorantraniliprole, beta-cyfluthrin, lambda-cyhalothrin, tefluthrin, fipronil and abamectin and component (II) is one or more plant activator(s), provided that:
a) if component (II) is acibenzolar-S-methyl then component (I) is not solely thiamethoxam,
b) if component (II) is acibenzolar-S-methyl or harpin then component (I) is not solely abamectin or a mixture of abamectin with one of clothianidin, imidacloprid or thiamethoxam,
c) if component (II) is acibenzolar-S-methyl or harpin then component (I) is not solely thiodicarb or a mixture of thiodicarb with one of clothianidin, imidacloprid or thiamethoxam, and
d) if component (II) is acibenzolar-S-methyl or harpin then no plant growth regulator selected from the group comprising paclobutrazol, cyproconazole, uniconazole, tetcyclacis, trinexapac, gibberellic acid and gibberellic acid-3 is present.

2. A combination according to claim 1 wherein component (II) is one or more plant activator(s) selected from the group comprising acibenzolar-S-methyl, CGA 210007, benzoic acid, harpin, magnesium-dihydrojasmonate and salicylic acid.

3. A combination according to claim 1 wherein
(a) thiodicarb as component (I) and acibenzolar-S-methyl as component (II);
(b) clothianidin as component (I) and acibenzolar-S-methyl as component (II);
(c) imidacloprid as component (I) and acibenzolar-S-methyl as component (II);
(d) acetamiprid as component (I) and acibenzolar-S-methyl as component (II);
(e) thiacloprid as component (I) and acibenzolar-S-methyl as component (II);
(f) chlorantraniliprole as component (I) and acibenzolar-S-methyl as component (II);
(g) beta-cyfluthrin as component (I) and acibenzolar-S-methyl as component (II);
(h) lambda-cyhalothrin as component (I) and acibenzolar-S-methyl as component (II);
(j) tefluthrin as component (I) and acibenzolar-S-methyl as component (II);
(k) fipronil as component (I) and acibenzolar-S-methyl as component (II);
(m) thiodicarb as component (I) and harpin as component (II);
(n) clothianidin as component (I) and harpin as component (II);
(o) imidacloprid as component (I) and harpin as component (II);
(p) acetamiprid as component (I) and harpin as component (II);
(q) thiacloprid as component (I) and harpin as component (II);
(r) chlorantraniliprole as component (I) and harpin as component (II);
(s) beta-cyfluthrin as component (I) and harpin as component (II);
(t) lambda-cyhalothrin as component (I) and harpin as component (II);
(u) tefluthrin as component (I) and harpin as component (II);
(v) fipronil as component (I) and harpin as component (II); or
(w) thiamethoxam as component (I) and harpin as component (II).

4. A combination according to claim 1 wherein the combination further comprises thiamethoxam and/or abamectin.

5. A combination according to claim 1 wherein the combination further comprises one or more fungicide(s).

6. A combination according to claim 1 wherein the combination further comprises one or more fungicide(s) selected from the group comprising phenylpyrrole fungicides, phenylamide fungicides, strobilurin fungicides and triazole fungicides, and mixtures thereof.

7. A combination according to claim 1 wherein the combination further comprises one or more fungicide(s) selected from the group comprising (a) fludioxonil, (b) metalaxyl, (c) mefenoxam, (d) azoxystrobin, (e) difenoconazole and/or (f) myclobutanil.

8. A combination according to claim 1 in the form of a plant propagation material treating pesticidal composition.

9. A method of controlling or preventing pest and/or pathogenic damage in a plant propagation material, a plant, and/or plant organs that grow at a later point in time, which comprises applying on the plant, part of the plant, or the surrounding area thereof the combination, as defined in claim 1, in any desired sequence or simultaneously.

10. A method of protecting a plant propagation material, a plant, and/or plant organs that grow at a later point in time against pest and/or pathogenic damage, which comprises applying to the plant, part of the plant, or the surrounding area thereof the combination, as defined in claim 1, in any desired sequence or simultaneously.

11. A method of improving the growing characteristics of a plant, and/or plant organs that grow at a later point in time, which comprises applying to the plant, part of the plant, or the surrounding area thereof the combination, as defined in claim 1, in any desired sequence or simultaneously.

12. A method according to claim 9, wherein the active ingredient components of the combination are applied simultaneously.

13. A method according to claim 9, wherein the combination is applied on plant propagation material.

14. A plant propagation material treated with the combination defined in claim 1.
